(19) United States
(12) Patent Application Publication
(10) Pub. No.: US 2008/0132603 A1
(43) Pub. Date: Jun. 5, 2008

(54) POLYMERIZABLE DENTAL MATERIAL BASED ON METHYL METHACRYLATES AND SUITABLE FOR THE PRODUCTION OF PLASTICS FOR DENTURES

(75) Inventors: KARL-HEINZ RENZ, FRANKFURT (DE); NOVICA SAVIC, RANSTADT (DE); KEVIN KERSCHER, FRIEDRICHSDORF (DE)

Correspondence Address: NORRIS, MCLAUGHLIN & MARCUS, P.A., 875 THIRD AVE, 18TH FLOOR, NEW YORK, NY, 10022, US

(73) Assignee: HERAEUS KULZER GMBH, HANAU (DE)

(21) Appl. No.: 11/942,329

(22) Filed: Nov. 19, 2007

(30) Foreign Application Priority Data
Nov. 20, 2006 (DE) 10 2006 054 879.5

(51) Int. Cl. A61K 6/083; C08L 33/06

(52) U.S. Cl. 523/115; 525/228

(57) ABSTRACT

A polymerizable dental material formed of
a liquid component containing
   A) at least one liquid methyl methacrylate monomer component,
   B) at least
      one acrylated or methacrylated butadiene oligomer or acrylated or methacrylated butadiene polymer and/or
      an acrylated or methacrylated acrylonitrile-butadiene oligomer or acrylated or methacrylated acrylonitrile-butadiene polymer,
         and optionally a powder component containing a polymer powder or a bead polymer based on methacrylate.

The invention relates to polymerizable dental materials based on methyl methacrylates and suitable for the production of plastics for dentures.

### BACKGROUND

Various materials are available for the production of full dentures, partial dentures, braces etc. for wearing in the mouth:
1. Thermally polymerizing plastics (1 or 2 components). These exhibit a very high thermally induced volume shrinkage leading to an inaccurate fit.
2. Self-polymerizing plastics (2 components).
3. Light polymerizing plastics (1 or 2 components).
4. Thermoplastic plastics (1 component), such materials are relatively difficult to process prosthodontically.
5. Microwave-curing plastics (1 or 2 components). In this case, too, a very high thermally-induced volume shrinkage leads to inaccurate fit.

Dentures made of the materials mentioned under 1 to 5 easily break when dropped or otherwise handled carelessly. Consequently, efforts have been made for some considerable time to make plastics for prosthodontic purposes fracture resistant. The undesired tendency to break is eliminated e.g. by using so-called high impact¹ plastics: ¹The term high impact is explained in further detail in ISO 1567—Denture Base Materials. Accordingly, the plastic for dentures is a high impact denture base material if its impact resistance according to ISO 1567 (based on Charpy) exceeds a value of 2 kJ/m².
6. Thermally polymerizing high impact plastics (1 or 2 components).

However, these again exhibit the undesired high thermally induced volume shrinkage leading to inaccurate fit.

Plastics for dentures have been introduced on the market which have solved the problem of shrinkage by the addition of butadiene-styrene rubber (EP 1 702 633 A2).

This technology has the disadvantage that the plastics thus obtained always bring about a certain turbidity. Even if the particle size of the rubber is smaller than the range of the wave-length of visible light an opalescent impression remains.

In an article by Kerby et al.—“Fracture toughness of modified dental resins” [J. of Oral Rehabilitation 30, 780-4 (2003)]—the use of methacrylate terminated 1,3-butadiene-acrylonitrile-acrylic acid terpolymer in dental material, particularly based on TEGDMA (triethylene glycol dimethacrylate) is described. The terpolymer is available under the trade name Hycar Reactive Liquid Polymer 1300×33. The publication does not dwell on the optical properties.

According to DE 196 17 876 A1, polysiloxane graft copolymers are used as fracture toughness modifiers: a polysiloxane graft copolymer which has a core of elastomeric polysiloxane and a sheath of non-elastomeric polymer and/or (2) a polysiloxane with (meth)acrylic groups.

U.S. Pat. No. 5,182,332 describes the use of (meth)acrylate grafted butadiene rubbers as additive to denture base material. It consists of the above-mentioned high impact denture plastics for dentures commonly used on the market. However, using the materials described therein, no transparent materials can be achieved. In U.S. Pat. No. 5,182,332, solid multi-layer rubbers are, moreover, preferably used (compare claim 1).

Two-component powder-liquid systems usually consist mainly of methacrylates. Methacrylates are available in powder and liquid form. Nowadays, the polymer powder is usually a bead polymer. It is mixed with liquid monomer in a weight ratio of 2.5 3:1. After a swelling period, a paste is obtained which can be pressed, cast or moulded. Typical compositions of this type have been described e.g. in DE 737 058 A and DE 37 25 502 A1.

The task arises of providing a polymerizable dental material based on methyl methacrylates and suitable for the production of plastics for dentures by means of which the above-described disadvantages can be entirely or partially avoided or its properties are improved.

### SUMMARY OF THE INVENTION

Surprisingly enough it has been found that acrylated or methacrylated butadiene oligomers and/or acrylonitrile oligomer or acrylated or methacrylated butadiene polymers and/or acrylonitrile polymers cause advantageous properties also in systems based on methyl methacrylate (MMA), in particular if they are added as liquid polymers (oligomers). Trans-parent products are obtained which, after curing, exhibit a transparency of >70% with a layer thickness of 3 mm. Moreover, it is advantageous that, after curing, the fracture toughness, fracture energy and impact resistance increase with a proportion of only approx. 1%—with simultaneously constant flexural strength and flexural modulus values.

This results in an increased fracture toughness during the useful life/application of the polymerized dental material.

The following, in particular, can be used as modifying agents:
methacrylated butadiene oligomer,
acrylated butadiene oligomer,
methacrylated acrylonitrile butadiene oligomer,
acrylated acrylonitrile butadiene oligomer,
further acrylated/methacrylated oligomers/polymers containing butadiene and/or acrylonitrile.

Using a methacrylated acrylonitrile-butadiene oligomer is particularly preferred.

### DETAILED DESCRIPTION

The invention thus relates to a polymerizable dental material comprising:

a liquid component containing
A) at least one liquid methyl methacrylate monomer component,
B) at least
   one acrylated or methacrylated butadiene oligomer or acrylated or methacrylated butadiene polymer and/or
   an acrylated or methacrylated acrylonitrile-butadiene oligomer or acrylated or methacrylated acrylonitrile-butadiene polymer,
      a powder component containing a polymer powder or a bead polymer.

The material has a light transmittance (transparency) in the visible range, after curing, of >70% with a layer thickness of 3 mm.

In addition, one or several substances selected from the group consisting of further monomers, fillers, pigments, stabilizers, regulators, antimicrobial additives, UV-absorbers, thixotroping agents, catalysts and crosslinking agents may be contained therein.

As further monomers, the monomers common in the dental sector can be considered as suitable:

Examples are free radical polymerizable monofunctional monomers such as mono(meth)acrylates, methyl (meth)acrylate, ethyl (meth)acrylate, butyl (meth)acrylate, benzyl (meth)acrylate, furfuryl (meth)acrylate or phenyl (meth)acrylate, difunctional or polyfunctional monomers such as difunctional or polyfunctional acrylates or methacrylates, e.g. bisphenol-A di(meth)acrylate, bis-GMA (an addition product from methacrylic acid and bisphenol-A diglycidyl ether), UDMA (an addition product from 2-hydroxyethyl methacrylate and 2,2,4-hexamethylene diisocyanate), diethylene glycol di(meth)acrylate-, triethylene glycol di(meth)acrylate or tetraethylene glycol di(meth)acrylate, decane diol di(meth)acrylate, dodecane diol di(meth)acrylate, hexyl decane diol di(meth)acrylate, trimethylol propane tri(meth)acrylate, pentaerythritol tetra(meth)acrylate and butane diol di(meth)acrylate, ethylene glycol di(meth)acrylate, polyethylene glycol di(meth)acrylates, ethoxylated/propoxylated bisphenol-A di(meth)acrylates.

As fillers, pyrogenic or precipitated silicic acids, dental glass such as aluminosilicate glass or fluoroaluminosilicate glass, strontium silicate, strontium borosilicate, lithium silicate, lithium aluminosilicate, layer silicates, zeolites, amorphous spherical fillers based on oxide or mixed oxide (SiO₂, ZrO₂ and/or TiO₂), metal oxides with primary particle sizes of approx. 40 to 300 nm, chip polymers with a particle size of 10-100 μm (compare R. Janda, Kunststoffverbundsysteme (polymer composites), VCH Verlagsgesellschaft, Weinheim, 1990, page 225 ff.) or their mixtures, for example, can be considered as suitable. Moreover, reinforcing agents such as glass fibers, polyamide fibers or carbon fibers can be incorporated.

As a rule, the fillers are used in amounts of 0 to 80% by wt., preferably 0 to 3% by wt., based on the total denture plastic composition and/or the sum total of the components A and B.

As regulator for adjusting the molecular weight, the following can, for example, be considered as suitable:

TGEH: thioglycolic acid-2-ethylhexyl ester,

t-DDM: tert-dodecyl mercaptan,

GDMA: Glycol dimercaptoacetate.

Examples of Initiators are

LPO: dilauroyl peroxide,

BPO: dibenzoyl peroxide,

t-BPEH: tert.-butyl per-2-ethyl hexanoate,

ADMV: 2,2′-azobis(2,4-dimethyl valeronitrile),

AIBN: 2,2′-azobis-(isobutyronitrile),

DTBP: di-tert-butyl peroxide.

Suitable stabilizers are e.g. hydroquinone monomethylether or 2,6-di-tert-butyl-4-methylphenol (BHT).

In addition, the denture base materials according to the invention may contain other usual additives e.g. from the group of antimicrobial additives, UV absorbers, thixotroping agents, catalysts and crosslinking agents.

As a rule, such other additives—such as pigments, stabilizers and regulators—are used in relatively small quantities, in total 0.01 to 3.0, in particular 0.01 to 1.0% by wt., based on the total mass of the material.

Curing of the composition preferably takes place by redox-induced free radical polymerization at room temperature or slightly elevated temperature at a slight pressure in order to avoid blister formation.

Redox initiator combinations, for example, such as combinations of benzoyl or lauryl peroxide with N,N-dimethyl sym. xylidine or N,N-dimethyl-p-toluidine are used as initiators for the polymerization carried out at room temperature. A combination of barbituric acids in combination with copper ions and chloride ions and the above-mentioned peroxides is a particularly preferred initiator system. This system is characterized by a high color stability.

The materials according to the invention are preferably used in the dental sector, above all for the production of dentures or orthodontic devices for correcting the position of teeth. Further possibilities for use, however, arise also in all areas in which a highly impact resistant moulded body has to be produced individually, e.g. in the case of
bone cements with an improved impact resistance,
applications in the field of veterinary medicine where impact resistance needs to be high, e.g. hoof repair material or tooth replacement for animals.

The proportion of the component in composition A) according to the invention is preferably more than 20% by wt., preferably >20 to 99% by wt., in particular >20 to 98% by wt., more particularly 20 to 50% by wt.

The proportion of component B) is preferably more than 1% by wt., and/or up to 99%, in particular 2-80% by wt., more particularly 50-80% by wt.

The materials according to the invention are suitable in particular for use in the dental sector for the production of dentures. Further possibilities exist in all areas in which a fracture resistant moulded body has to be produced individually, e.g. in the case of
bone cement with improved impact resistance,
applications in the field of veterinary medicine in the case of which the impact resistance must be high, e.g. hoof repair material or tooth replacement for animals,
orthodontic devices for correcting the position of teeth.

### EXAMPLE

The following table of experiments shows that the modulus of elasticity, above all, changes in a highly advantageous manner in the case of the addition according to the invention of the copolymers. The basis for the experiments was a conventional denture material PalaX-press (powder/liquid based on methacrylate) cold polymerizing² with dibenzoyl peroxide, the modifier having been homogenized with the liquid.

The results of the investigation of the polymerized samples are as follows:

² Cold polymers (also “warm polymers”) (commercial names: Trigon 40, PalaXpress, Castodon it) exhibit a special catalyst system which, although initiating the polymerisation, delays it simultaneously to such an extent that a sufficiently long processing time is guaranteed. These plastics can therefore also be cast before they have partially swollen, thus having a universal processing range (casting technique, injection technique, total endoprosthetics, partial prosthetics).

| Proportion of | | Bending | Flexural | Impact | Fracture | Fracture |
|---|---|---|---|---|---|---|
| modifier | | strength | modulus | resistance | toughness | energy |
| [% by wt.] | Art Modifier | in MPa | in MPa | in kJ/m² | in MPa · m^{1/2} | in J/m² |
| 0.0 | — | 67 | 2017 | 0.95 | 1.53 | 239 |
| 2.0 | Methacrylated butadi- | 63 | 1867 | 1.08 | 1.98 | 515 |
| | ene oligomer | | | | | |
| 2.0 | Acrylated butadiene | 65 | 1953 | 1.17 | 1.83 | 496 |
| | oligomer | | | | | |
| 2.0 | Methacrylated acry- | 68 | 2020 | 1.55 | 2.5 | 926 |
| | lonitrile-butadiene | | | | | |
| | oligomer | | | | | |
| 1.0 | Methacrylated acry- | 69 | 2054 | 1.36 | 2.22 | 519 |
| | lonitrile-butadiene | | | | | |
| | oligomer | | | | | |
| 2.4 | Methacrylated acry- | 66 | 1975 | 1.65 | 2.49 | 1042 |
| | lonitrile-butadiene | | | | | |
| | oligomer | | | | | |
| 3.6 | Methacrylated acry- | 64 | 1896 | 1.84 | 2.59 | 1138 |
| | lonitrile-butadiene | | | | | |
| | oligomer | | | | | |
| 2.4 | Methacrylated acry- | 67 | 1957 | 1.44 | 2.42 | 970 |
| | lonitrile-butadiene | | | | | |
| | oligomer | | | | | |
| 4.8 | Methacrylated acry- | 60 | 1723 | 1.65 | 2.26 | 979 |
| | lonitrile-butadiene | | | | | |
| | oligomer | | | | | |

We claim:

1. Polymerizable dental material comprised of:
I a liquid component comprising
A) at least one liquid methyl methacrylate monomer component,
B) at least
one acrylated or methacrylated butadiene oligomer or acrylated or methacrylated butadiene polymer and/or
an acrylated or methacrylated acrylonitrile-butadiene oligomer or acrylated or methacrylated acrylonitrile-butadiene polymer,
II a powder component comprising a polymer powder or a bead polymer based on methacrylate,
and having, after curing, a transparency of >70% at a layer thickness of 3 mm.

2. Polymerizable dental material according to claim 1, further comprising one or several substance(s) selected from the group consisting of further monomers, fillers, pigments, stabilizers, regulators, antimicrobial additives, UV absorbers, thixotroping agents, catalysts and crosslinking agents.

3. Polymerizable dental material according to claim 1, wherein the proportion of component B) is >1% by wt.

4. Polymerizable dental material according to claim 3, wherein said proportion of component B) is >1 to 99% by wt.

5. Polymerizable dental material according to claim 5, wherein said proportion of component B) is 2 to 80% by wt.

6. Polymerizable dental material according to claim 5, wherein said proportion of component B) is 50 to 80% by wt.

7. Polymerizable dental material according to claim 1, wherein the proportion of component A) is >20% by wt.

8. Polymerizable dental material according to claim 7, wherein the proportion of component A) is >20 to 99% by wt.

9. Polymerizable dental material according to claim 8, wherein the proportion of component A) is >20 to 50% by wt.

10. A bone cement with improved impact resistance,
a hoof repair material or dental prosthesis for animals, or an
orthodontic device for correcting the position of teeth,
comprising the polymerizable dental material of claim 1.
